SEQUENCE LISTING (1 sequence)

SEQ ID NO: 1 - protein, 450 aa, synthetic construct
METDTLLLWVLLLWVPGSTGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSMDRLTSSFLLLIVPAYVLSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0161412 A1
(43) Pub. Date: May 22, 2025

(54) COMBINATION THERAPIES FOR MULTIPLE MYELOMA

(71) Applicants: NantCell, Inc., Culver City, CA (US); ImmunityBio, Inc., Culver City, CA (US)

(72) Inventors: John Lee, Culver City, CA (US); Peter Rhode, Miami, FL (US)

(21) Appl. No.: 19/021,950

(22) Filed: Jan. 15, 2025

Related U.S. Application Data
(63) Continuation of application No. 17/292,701, filed on May 10, 2021, now abandoned.
(60) Provisional application No. 62/760,772, filed on Nov. 13, 2018.

(51) Int. Cl. A61K 38/20; A61K 38/17; A61K 40/11; A61K 40/41; A61K 40/42; C07K 16/28

(52) U.S. Cl. CPC A61K 38/2086; A61K 38/1793; A61K 40/11; A61K 40/418; A61K 40/421; C07K 16/2818; A61K 2239/31; A61K 2239/38; C07K 2317/21; C07K 2317/24; C07K 2319/30

(57) ABSTRACT

Compositions and methods are provided to treat and prevent cancers, such as myelomas, and include adoptive cell therapies in combination with an IL-15 superagonist and one or more chemotherapeutic agents.

### RELATED APPLICATIONS

The present application claims the benefit of U.S. Provisional application No. 62/760,772, filed on Nov. 13, 2018. The entire contents of the aforementioned application are incorporated herein by reference.

### FIELD OF THE INVENTION

Compositions in the prevention and treatment of cancers, such as myelomas, combine adoptive cell therapies with an IL-15 superagonist and chemotherapeutic agents.

### BACKGROUND

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy. Cancer can occur in any tissue or organ of the body. Plasma cell neoplasms, including multiple myeloma, “Solitary” myeloma of bone, extramedullary plasmacytoma, plasma cell leukemia, macroglobulinemia (including Waldenstrom's macroglobulinemia), heavy-chain disease, primary amyloidosis, monoclonal gammopathy of unknown significance (MGUS) are associated with increased expression of immunoglobulins. Chronic lymphocytic leukemia (CLL), a non-plasma cell neoplasm, is also associated with high levels of immunoglobulin expression.

Myelomas are tumors of plasma cells derived from a single clone, which typically originates in secondary lymphoid tissue and then migrates into and resides in bone marrow tissue. Myelomas commonly affect the bone marrow and adjacent bone structures, with primary symptoms of bone pain and pathological fractures or lesions (osteolytic bone lesions), abnormal bleeding, anemia and increased susceptibility to infections. Advanced stages of the disease include renal failure, skeletal deformities, compaction of the spinal cord, and hypercalcemia. Myeloma affects bone cells by inducing osteoclast resorption of bone, hence decimating bone structure and increasing calcium concentration in plasma.

Traditional treatment regions for myeloma and multiple myelomas (henceforth referred to as “myeloma”) consist of chemotherapy, radiation therapy, and surgery. In addition, bone marrow transplantation is recommended for patients who are otherwise in good health. The cure rate for patient's approaches 30%, and is the only method known that can cure myelomas. However, for individuals who are older or cannot tolerate bone marrow transplantation procedures, chemotherapy is most appropriate.

Recently, advances in multiple myeloma therapies such as the introduction of autologous stem cell transplantation (ASCT) (S. Mahajan et al., Ther Adv Hematol. 2018 May; 9 (5): 123-133) and the availability of thalidomide, lenalidomide (immunomodulatory drugs or IMiDs), and bortezomib have changed the management of these patients and have allowed an increase in overall survival (OS) (Kristinsson et al., J. Clin. Oncol., 25:1993-1999 (2007); Brenner et al., Blood, 111:2521-2526 (2008); and Kumar et al., Blood, 111:2516-2520 (2008)). Patients younger than 60 years have a 10 year survival probability of 30% (Raab et al., Lancet, 374:324-339 (2009)). Thalidomide (Rajkumar et al., J. Clin. Oncol., 26:2171-2177 (2008)), lenalidomide (Rajkumar et al., Lancet Oncol., 11:29-37 (2010)); or bortezomib (Harousseau et al., J. Clin. Oncol., 28:4621-4629 (2010)), in combination with dexamethasone as part of an induction therapy regimen before ASCT has led to rates of nearly complete response (CR) rates of 8, 15 and 16%, respectively; whereas three-drug induction schedules of bortezomib-dexamethasone plus doxorubicin (Sonneveld et al., Blood (ASH Annual Meeting Abstracts), 116:23 (2010)), cyclophosphamide (Reeder et al., Leukemia, 23:1337-1341 (2009)), thalidomide (Cavo et al., Lancet, 376:2075-2085 (2010)); or lenalidomide (Richardson et al., Blood, 116:679-686 (2010)), permits achievement rates of nearly CR of 7, 39, 32 and 57%, respectively.

Immunostimulatory monoclonal antibodies (mAb) represent a new strategy in cancer immunotherapy to potentiate the immune responses of the host against the malignancy (Melero et al., Nat. Rev. Cancer, 7:95-106 (2007)). In spite of the promising anti-tumor efficacy of several monoclonal antibodies, many tumors are refractory to treatment with a single antibody (Wilcox et al., J. Clin. Invest., 109:651-659 (2002); Verbrugge et al., Cancer Res., 72:3163-3174 (2012)), and combinations of two or more antibodies may be needed.

However, despite these advances, almost all multiple myeloma patients relapse.

### SUMMARY

Embodiments of the invention are directed to compositions for the treatment of cancer. Methods of treatment comprise administration of the compositions to a subject to prevent or treat the cancer.

In one aspect, a method of treating cancer is provided, comprising administering to a subject an effective amount of i) an adoptive cell therapy, ii) an IL-15:IL-15Rα complex, and iii) at least one chemotherapeutic agent. In certain embodiments, the IL-15/IL15Rα complex is an IL-15N72D:IL-15RαSu/Fc complex comprising a dimeric IL-15RαSu/Fc and two IL-15N72D molecules. In certain embodiments, the adoptive cell therapy comprises hematopoietic stem cell transplantation, donor leukocyte infusion, adoptive transfer of natural killer cells (NK), T cells, B cells, chimeric antigen receptor-T cells (CAR-T), chimeric antigen receptor natural killer cells (CAR-NK) or combinations thereof. In certain embodiments, the adoptive cell therapy comprises transfer of allogeneic, autologous, syngeneic, related, unrelated, HLA-matched, HLA-mismatched or haploidentical cells. In certain embodiments, the adoptive cell therapy comprises NK cells. In certain embodiments, the cancer comprises: myeloma, multiple myeloma, smoldering myeloma, relapsed or refractory multiple myeloma, hematological cancer, chronic myelogenous leukemia, acute lymphocytic leukemia, acute myelogenous leukemia, acute lymphoblastic leukemia, myelodysplasia, mantle cell lymphoma, B cell non-Hodgkin lymphoma, Hodgkin's lymphoma, chronic lymphocytic leukemia, lymphoma, non-Hodgkin's lymphomas (NHL), chronic lymphocytic leukemia, follicular lymphoma, mantle cell lymphoma or diffuse large B-cell lymphoma. In certain embodiments, the cancer comprises myeloma, multiple myeloma, or smoldering myeloma. In certain embodiments, the chemotherapeutic agent comprises: anti-CS1 antibody (Elotuzumab), bortezomib, lenalidomide (Revlimid), dexamethasone, melphalan, vincristine (Oncovin), cyclophosphamide (Cytoxan), etoposide (VP-16), doxorubicin (Adriamycin), liposomal doxorubicin (Doxil), bendamustine (Treanda), anti-PD1 antibody (nivolumab or pembrolizumab) or combinations thereof. In certain embodiments, the chemotherapeutic agent comprises anti-CS1 antibody (Elotuzumab).

In another aspect, a method of treating a myeloma is provided, the method comprising administering to a subject an effective amount of: i) an adoptive cell therapy, ii) an IL-15:IL-15Rα complex, and iii) at least one chemotherapeutic agent. In certain embodiments, the IL-15/IL15Rα complex is an IL-15N72D:IL-15RαSu/Fc complex comprising a dimeric IL-15RαSu/Fc and two IL-15N72D molecules. In certain embodiments, the adoptive cell therapy comprises hematopoietic stem cell transplantation, donor leukocyte infusion, adoptive transfer of natural killer cells (NK), T cells, B cells, chimeric antigen receptor-T cells (CAR-T), chimeric antigen receptor natural killer cells (CAR-NK) or combinations thereof. In certain embodiments, the chemotherapeutic agent comprises: anti-CS1 antibody (Elotuzumab), bortezomib, lenalidomide (Revlimid), dexamethasone, melphalan, vincristine (Oncovin), cyclophosphamide (Cytoxan), etoposide (VP-16), doxorubicin (Adriamycin), liposomal doxorubicin (Doxil), bendamustine (Treanda), anti-PD1 antibody (nivolumab or pembrolizumab) or combinations thereof. In certain embodiments, the chemotherapeutic agent is elotuzumab.

In certain embodiments, the at least one chemotherapeutic agent is administered prior to, simultaneously with, sequentially to the adoptive cell therapy, or any combination thereof. In certain embodiments, the at least one chemotherapeutic agent is administered prior to the administration of the adoptive cell therapy. In certain embodiments, the at least one chemotherapeutic agent is administered concomitantly with the administration of the adoptive cell therapy. In certain embodiments, the at least one chemotherapeutic agent is administered after the administration of the adoptive cell therapy.

In certain embodiments, the therapeutically effective amount of IL-15N72D:IL-15RαSu/Fc complex is administered multiple times per week, such as one or twice or more per week. In certain embodiments, the therapeutically effective amount of IL-15N72D:IL-15RαSu/Fc complex is administered daily. In certain embodiments, the therapeutically effective amount of IL-15N72D:IL-15RαSu/Fc complex is between 0.1 μg/Kg and 100 mg/Kg. In embodiments, the IL-15N72D:IL-15RαSu/Fc complex stimulates proliferation or activation of adoptively transferred cells.

In certain embodiments, the method of treating a cancer or myeloma further comprises administering an immunomodulatory agent, anti-anemia agents, radiation therapy, corticosteroids, cytokines, chemokines or combinations thereof.

In certain embodiments, the adoptive cell therapy comprises NK cells. In certain embodiments, NK cells are obtained from one or more sources comprising ascites, peritoneum, lymph, blood, plasma or combinations thereof.

In another aspect a pharmaceutical composition is provided, the pharmaceutical composition comprising an effective amount of adoptive cell therapy, an IL-15/IL-15Rα fusion complex, a chemotherapeutic agent or combinations thereof. In certain embodiments, the IL-15/IL-15Rα fusion complex is IL-15N72D:IL-15RαSu/Fc. In certain embodiments, the IL-15N72D:IL-15RαSu/Fc complex comprises a dimeric IL-15RαSu/Fc and two IL-15N72D molecules. In certain embodiments, the chemotherapeutic agent comprises: anti-CS1 antibody (Elotuzumab), bortezomib, lenalidomide (Revlimid), dexamethasone, melphalan, vincristine (Oncovin), cyclophosphamide (Cytoxan), etoposide (VP-16), doxorubicin (Adriamycin), liposomal doxorubicin (Doxil), bendamustine (Treanda), anti-PD1 antibody (nivolumab or pembrolizumab) or combinations thereof.

In another aspect, a pharmaceutical composition comprises an effective amount of IL-15/IL-15Rα and a chemotherapeutic agent comprising: anti-CS1 antibody (Elotuzumab), bortezomib, lenalidomide (Revlimid), dexamethasone, melphalan, vincristine (Oncovin), cyclophosphamide (Cytoxan), etoposide (VP-16), doxorubicin (Adriamycin), liposomal doxorubicin (Doxil), bendamustine (Treanda), anti-PD1 antibody (nivolumab or pembrolizumab) or combinations thereof. In certain embodiments, the fusion complex is IL-15N72D:IL-15RαSu/Fc. In certain embodiments, the IL-15N72D:IL-15RαSu/Fc complex comprises a dimeric IL-15RαSu/Fc and two IL-15N72D molecules. In certain embodiments, the chemotherapeutic agent is an anti-CS1 antibody (Elotuzumab).

In certain embodiments, a pharmaceutical composition is administered systemically, intravenously, subcutaneous, intramuscularly, intravesically, or by instillation.

In certain embodiments, the administration of the adoptively transferred cells and the pharmaceutical composition results in prolonged survival of said subject compared to an untreated subject.

In another aspect, a kit for treating cancer is provided, the kit comprising an adoptive cell therapy, an IL-15/IL15Rα complex, at least one chemotherapeutic agent and directions for the use of the kit for the treatment of cancer. In certain embodiments, the adoptive cell therapy comprises hematopoietic stem cells, donor leukocytes, T cells, or natural killer (NK) cells. In certain embodiments, the IL-15/IL15Rα complex is an IL-15N72D:IL-15RαSu/Fc complex comprising a dimeric IL-15RαSu/Fc and two IL-15N72D molecules. In certain embodiments, the chemotherapeutic agent is elotuzumab.

In yet another aspect, a kit for treating myeloma is provided, the kit comprising an adoptive cell therapy, an IL-15/IL15Rα complex, at least one chemotherapeutic agent and directions for the use of the kit for the treatment of myeloma. In certain embodiments, the adoptive cell therapy comprises hematopoietic stem cells, donor leukocytes, T cells, or natural killer (NK) cells. In certain embodiments, the IL-15/IL15Rα complex is an IL-15N72D:IL-15RαSu/Fc complex comprising a dimeric IL-15RαSu/Fc and two IL-15N72D molecules. In certain embodiments, the chemotherapeutic agent is elotuzumab.

In certain embodiments, the chemotherapeutic agent, for example, elotuzumab is administered at a dosage of about 0.01 to about 100 mg/Kg, or from about 0.01 to about 90 mg/Kg, or from about 0.01 mg/Kg to about 80 mg/Kg or to about 70 mg/Kg or to about 60 mg/Kg or to about 50 mg/Kg or to about 40 mg/Kg or to about 30 mg/Kg or to about 20 mg/Kg or to about 20 mg/Kg or to about 10 mg/Kg or to about 5 mg/Kg. 10 mg/Kg, or about 20 mg/Kg, or about 30 mg/Kg or about 40 mg/Kg or about 50 mg/Kg, once every week, or twice every week.

Any compositions or methods provided herein can be combined with one or more of any of the other compositions and methods provided herein.

Other aspects are described infra.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The articles the terms “a”, “an”, and “the” are understood to be singular or plural and are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “cancer therapy” refers to a therapy useful in treating cancer. Examples of anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN™), anti-CD20 antibodies, an epidermal growth factor receptor (EGFR) antagonist (e.g., a tyrosine kinase inhibitor), HER1/EGFR inhibitor (e.g., erlotinib (TARCEVA™)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

In one embodiment, the effective amount is administered to a patient that has been diagnosed with cancer. The effective amount can result in the prevention of the development, recurrence, or onset of cancer and one or more symptoms thereof, to enhance or improve the efficacy of another therapy, reduce the severity, the duration of cancer, ameliorate one or more symptoms of cancer, prevent the advancement of cancer, cause regression of cancer, and/or enhance or improve the therapeutic effect(s) of another therapy. “Effective amount” also refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. In an embodiment of the invention, the amount of a therapy is effective to achieve one, two, three, or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, (12) an increase in the number of patients in remission, (13) an increase in the length or duration of remission, (14) a decrease in the recurrence rate of cancer, (15) an increase in the time to recurrence of cancer, and (16) an amelioration of cancer-related symptoms and/or quality of life.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5,000, about 3,000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

By “reduces” is meant a negative alteration of at least 5%, 10%, 25%, 50%, 75%, or 100%.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “subject” is meant a mammal, including, but not limited to, a human or non-human mammal, such as a bovine, equine, canine, ovine, or feline. The subject is preferably a mammal in need of such treatment, e.g., a subject that has been diagnosed with B cell lymphoma or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “treating” and “treatment” as used herein refer to the administration of an agent or formulation to a clinically symptomatic individual afflicted with an adverse condition, disorder, or disease, so as to affect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated. Agents or formulations used in treatment may comprise cells or tissues.

Treatment of patients with neoplasia may include any of the following: Adjuvant therapy (also called adjunct therapy or adjunctive therapy) to destroy residual tumor cells that may be present after the known tumor is removed by the initial therapy (e.g. surgery), thereby preventing possible cancer reoccurrence; neoadjuvant therapy given prior to the surgical procedure to shrink the cancer; induction therapy to cause a remission, typically for acute leukemia; consolidation therapy (also called intensification therapy) given once a remission is achieved to sustain the remission; maintenance therapy given in lower or less frequent doses to assist in prolonging a remission; first line therapy (also called standard therapy); second (or 3rd, 4th, etc.) line therapy (also called salvage therapy) is given if a disease has not responded or reoccurred after first line therapy; and palliative therapy (also called supportive therapy) to address symptom management without expecting to significantly reduce the cancer.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference.

### DETAILED DESCRIPTION

Embodiments of the invention include combination therapies in the prevention and treatment of cancer. In particular, these include the combination of an IL-15 superagonist with adoptive cell therapy and one or more chemotherapeutic agents.

### Adoptive Cell Therapy

Adoptive cell therapy (ACT) (including allogeneic and autologous hematopoietic stem cell transplantation (HSCT) and recombinant cell (i.e., CAR T) therapies) is the treatment of choice for many malignant disorders (for reviews of HSCT and adoptive cell therapy approaches, see, Rager & Porter, Ther Adv Hematol (2011) 2(6) 409-428; Roddie & Peggs, Expert Opin. Biol. Ther. (2011) 11(4):473-487; Wang et al. Int. J. Cancer. (2015)136, 1751-1768; and Chang, Y. J. and X. J. Huang, Blood Rev, 2013. 27(1): 55-62). Such adoptive cell therapies include, but are not limited to, allogeneic and autologous hematopoietic stem cell transplantation, donor leukocyte (or lymphocyte) infusion (DLI), adoptive transfer of tumor infiltrating lymphocytes, or adoptive transfer of T cells or NK cells (including recombinant cells, i.e., CAR T, CAR NK, gene-edited T cells or NK cells, see Hu et al., Acta Pharmacologica Sinica (2018) 39: 167-176, Irving et al. Front Immunol. (2017) 8: 267). Beyond the necessity for donor-derived cells to reconstitute hematopoiesis after radiation and chemotherapy, immunologic reconstitution from transferred cells is important for the elimination of residual tumor cells. The efficacy of ACT as a curative option for malignancies is influenced by a number of factors including the origin, composition and phenotype (lymphocyte subset, activation status) of the donor cells, the underlying disease, the pre-transplant conditioning regimen and post-transplant immune support (i.e., IL-2 therapy) and the graft-versus-tumor (GVT) effect mediated by donor cells within the graft. Additionally, these factors must be balanced against transplant-related mortality, typically arising from the conditioning regimen and/or excessive immune activity of donor cells within the host (i.e., graft-versus-host disease, cytokine release syndrome, etc.).

In certain embodiments, the adoptive cell therapy comprises hematopoietic stem cell transplantation, donor leukocyte infusion, adoptive transfer of natural killer cells (NK), T cells, B cells, chimeric antigen receptor-T cells (CAR-T), chimeric antigen receptor natural killer cells (CAR-NK) or combinations thereof.

Approaches utilizing adoptive NK cell therapy have become of significant interest. In patients receiving autologous HSCT, blood NK cell numbers recover very early after the transplant and the levels of NK cells correlate with a positive outcome (Rueff et al., 2014, Biol. Blood Marrow Transplant. 20, 896-899). Although therapeutic strategies with autologous NK cell transfer have had limited success due to a number of factors, adoptive transfer of ex vivo-activated allogeneic (or haplo-identical) NK cells has emerged as a promising immunotherapeutic strategy for cancer (Guillerey et al. 2016. Nature Immunol. 17: 1025-1036). The activity of these cells is less likely to be suppressed by self-MHC molecules compared to autologous NK cells. A number of studies have shown that adoptive therapy with haploidentical NK cells to exploit alloreactivity against tumor cells is safe and can mediate significant clinical activity in AML patients. Taking these findings further, recent studies have focused on optimizing ex vivo activation/expansion methods for NK cells or NK precursors (i.e., stem cells) and pre-transplant conditioning and post-transplant immune support strategies; use of NK cell lines or recombinant tumor-targeting NK cells; evaluation of combination therapies with other agents such as therapeutic antibodies, immunomodulatory agents (lenalidomide), and anti-KIR and checkpoint antibodies. In each case, these strategies could be complemented by the combination therapeutic approach of the invention, which has the capacity to augment NK cell proliferation and activation.

Natural Killer Cells: One of the major types of circulating mononuclear cells is that of the natural killer, or NK, cell (M. Manoussaka et al., Journal of Immunology 158:112-119, 1997). Originally defined based on their ability to kill certain tumors and virus-infected cells, NK cells are now known as one of the components of the early, innate immune system. In addition to their cytotoxic capabilities, NK cells serve as regulators of the immune response by releasing a variety of cytokines. In addition, the generation of complex immune responses is facilitated by the direct interaction of NK cells with other cells via various surface molecules expressed on the NK cells.

NK cells are derived from bone marrow precursors (O. Haller et al., Journal of Experimental Medicine 145:1411-1420, 1977). NK cells appear to be closely related to T cells, and the two cell types share many cell surface markers (M. Manoussaka et al., 1997). As noted above, these cell surface markers play a significant role in NK cell activity. For example, murine NK cells express specific antigens on their surfaces, such as asialo GM1, NK1, and NK2 antigens (D. See et al., Scand. J. Immunol. 46:217-224, 1997), and the administration of antibodies against these antigens results in depletion of NK cells in vivo (Id.).

Similarly to cytotoxic T lymphocytes (CTL), NK cells exert a cytotoxic effect by lysing a variety of cell types (Srivastava, S., Lundqvist, A. & Childs, R. W. Natural killer cell immunotherapy for cancer: a new hope. Cytotherapy 10, 775-783; 2008). These include normal stem cells, infected cells, and transformed cells. The lysis of cells occurs through the action of cytoplasmic granules containing proteases, nucleases, and perforin. Cells that lack MHC class I are also susceptible to NK cell-mediated lysis (H. Reyburn et al., Immunol. Rev. 155:119-125, 1997). In addition, NK cells exert cytotoxicity in a non-MHC restricted fashion (E. Ciccione et al., J. Exp. Med. 172:47, 1990; A. Moretta et al., J. Exp. Med. 172:1589, 1990; and E. Ciccione et al., J. Exp. Med. 175:709). NK cells can also lyse cells by antibody-dependent cellular cytotoxicity.

As noted above, NK cells mediate some of their functions through the secretion of cytokines, such as interferon γ (IFN-γ), granulocyte-macrophage colony-stimulating factors (GM-CSFs), tumor necrosis factor a (TNF-α), macrophage colony-stimulating factor (M-CSF), interleukin-3 (IL-3), and IL-8. NK cell cytotoxic activity is regulated through a balance of activating and inhibitory receptors that enables fine-tuned control of cytotoxic activity, preventing cytotoxicity against healthy cells, while maintaining effective cytotoxic capacity against tumor cells. Indeed, multiple studies have demonstrated the safety of adoptive NK cell transfer and clinical anti-cancer effects, highlighting the potential for NK cells as an effective cancer immunotherapy ((Parkhurst, M. R., et al. Clin Cancer Res 17, 6287-6297 (2011); Ruggeri, L. et al. Science 295, 2097-2100, (2002); Miller, J. S. et al. Blood 105, 3051-3057, (2005; Bachanova, V. et al. Blood 123, 3855-3863, (2014); Rubnitz, J. E. et al. J Clin Oncol 28, 955-959, (2010)). For example, cytokines including IL-2, IL-12, TNF-α, and IL-1 can induce NK cells to produce cytokines. IFN-α and IL-2 are strong inducers of NK cell cytotoxic activity (G. Trinichieri et al., Journal of Experimental Medicine 160:1147-1169, 1984; G. Trinichieri and D. Santoli, Journal of Experimental Medicine 147:1314-1333, 1977). The presence of IL-2 both stimulates and expands NK cells (K. Oshimi, International Journal of Hematology 63:279-290, 1996). IL-12 has been shown to induce cytokine production from T and NK cells, and augment NK cell-mediated cytotoxicity (M. Kobayashi et al., Journal of Experimental Medicine 170:827-846, 1989).

NK cells are involved in both the resistance to and control of cancer spread. Since the advent of the cancer immune surveillance concept, the adoptive transfer of immune cells, particularly T cells and natural killer (NK) cells, has emerged as a targeted method of harnessing the immune system against cancer (Kroemer, G., Senovilla, L., Galluzzi, L., Andre, F. & Zitvogel, L. Natural and therapy-induced immunosurveillance in breast cancer. Nat Med 21, 1128-1138, (2015)). NK cells have garnered immense attention as a promising immunotherapeutic agent for treating cancers. NK cells are critical to the body's first line of defense against cancer due to their natural cytotoxicity against malignant cells (Srivastava, S., et al., Cytotherapy 10, 775-783; 2008).

NK cells have been expanded from multiple sources, including peripheral blood and umbilical cord blood (CB) ((Denman, C. J. et al. Membrane-bound IL-21 promotes sustained ex vivo proliferation of human natural killer cells. PLoS One 7, e30264, (2012); Knorr, D. A. et al. Clinical-scale derivation of natural killer cells from human pluripotent stem cells for cancer therapy. Stem Cells Transl Med 2, 274-283, (2013); Shah, N. et al. Antigen presenting cell-mediated expansion of human umbilical cord blood yields log-scale expansion of natural killer cells with anti-myeloma activity. PLoS One 8, e76781, (2013); Woll, P. S. et al. Human embryonic stem cells differentiate into a homogeneous population of natural killer cells with potent in vivo antitumor activity. Blood 113, 6094-6101, (2009)). Ex vivo NK cell expansion methods have been developed using cytokines in combination with artificial antigen-presenting cells (aAPCs) as feeder cells ((Denman, C. J. et al. PLoS One 7, e30264, (2012); Berg, M. et al. Cytotherapy 11, 341-355, (2009); Gong, W. et al. Tissue Antigens 76, 467-475, (2010); Zhang, H. et al., J Immunother 34, 187-195, (2011)).

### IL-15 Superagonist

This IL-15 super agonist in combination with a soluble IL-15α receptor fusion protein (IL-15Rα-Fc) results in a protein complex (IL-15N72D/IL-15Rα-Fc) with highly potent IL-15 activity in vitro and in vivo.

In certain embodiments, an IL-15 receptor α/IgG1 Fc fusion protein (IL-15N72D:IL-15RαSu/Fc) can be administered as part of the adoptive cell therapy and can include one or more chemotherapeutic agents. N-803 comprises an IL-15 mutant with increased ability to bind IL-2Rβγ and enhanced biological activity (U.S. Patent No. 8,507, 222, incorporated herein by reference). This super-agonist mutant of IL-15 was described in a publication (Zu et al., 2009 J Immunol, 183:3598-3607, incorporated herein by reference). This IL-15 super-agonist in combination with a soluble IL-15α receptor fusion protein (IL-15RαSu/Fc) results in a fusion protein complex with highly potent IL-15 activity in vitro and in vivo (Han et al., 2011, Cytokine, 56: 804-810; Xu, et al., 2013 Cancer Res. 73:3075-86, Wong, et al., 2013, OncoImmunology 2:e26442). The IL-15 super agonist complex comprises an IL-15 mutant (IL-15N72D) bound to an IL-15 receptor a/IgG1 Fc fusion protein (IL-15N72D:IL-15RαSu/Fc) is referred to as “N-803.”

Pharmacokinetic analysis indicated that the fusion protein complex has a half-life of 25 hours following i.v. administration in mice. N-803 exhibits impressive anti-tumor activity against aggressive solid and hematological tumor models in immunocompetent mice. It can be administered as a monotherapy using a twice weekly or weekly i.v. dose regimen or as combinatorial therapy with an antibody. The N-803 anti-tumor response is also durable. Tumor-bearing mice that were cured after N-803 treatment were also highly resistant to re-challenge with the same tumor cells indicating that N-803 induces effective immunological memory responses against the re-introduced tumor cells.

The sequence for N-803 (IL-15N72D associated with a dimeric IL-15RαSu/Fc fusion protein) comprises SEQ ID NO: 1:

IL-15N72D protein sequence (with leader peptide)
METDTLLLWVLLLWVPGSTG-
[Leader peptide]
NWVNVISDLKKIEDLIQSMHIDATLYTESDVHPSCKVTAMKCFLLELQVI
SLESGDASIHDTVENLIILANDSLSSNGNVTESGCKECEELEEKNIKEFL
QSFVHIVQMFINTS
[IL-15N72D]
IL-15RαSu/Fc protein sequence (with leader
peptide)
MDRLTSSFLLLIVPAYVLS-
[Leader peptide]
ITCPPPMSVEHADIWVKSYSLYSRERYICNSGFKRKAGTSSLTECVLNKA
TNVAHWTTPSLKCIR-
[IL-15RαSu]
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK
[IgG1 CH2-CH3 (Fc domain)].

Accordingly, in certain embodiments, in certain embodiments, the method of treating cancer comprises administering to the patient, an effective amount of an adoptive cell therapy, a pharmaceutical composition comprising a therapeutically effective amount of an IL-15:IL-15Rα complex and/or at least one chemotherapeutic agent. The IL-15/IL15Rα complex is an IL-15N72D:IL-15RαSu/Fc complex (N-803) comprising a dimeric IL-15RαSu/Fc and two IL-15N72D molecules. In certain embodiments, a pharmaceutical composition comprising a therapeutically effective amount of at least one chemotherapeutic agent is also administered to the patient as part of a combination therapy.

In certain embodiments, a method of treating cancer comprises administering to a subject an effective amount of an adoptive cell therapy, a pharmaceutical composition comprising a therapeutically effective amount of an IL-15:IL-15Rα complex, at least one chemotherapeutic agent or a combination thereof. The IL-15/IL15Rα complex is an IL-15N72D:IL-15RαSu/Fc complex (N-803) comprising a dimeric IL-15RαSu/Fc and two IL-15N72D molecules. In certain embodiments, the cells, e.g. NK cells are contacted with the N-803 fusion protein complex. The ex vivo incubation of NK cells with the fusion protein complex results in induction of CIML NK cell exhibiting elevated activation markers, increased cytotoxicity against tumor cells and enhanced production of IFN-y. Additionally, the fusion protein complex is capable of activating human NK cell lines. Moreover, methods are provided for augmenting immune responses and treating neoplasia and infection disease by direct administration of the fusion protein complex of the invention or administration of immune cells activated by the fusion protein complex of the invention.

### Chemotherapeutic Agents

Myeloma cells express high levels of CS1 or SLAMF7 (also referred to as CD subset 2, CD319, CRACC, 19A, APEX-1, FOAP12, and 19A; GENBANK™ Accession No. NM_021181.3, Ref. Boles et al., Immunogenetics, 52:302-307 (2001); Bouchon et al., J. Immunol., 167:5517-5521 (2001); Murphy et al., Biochem. J., 361:431-436 (2002))), a cell-surface receptor that belongs to the signaling-lymphocytic-activation-molecule (SLAM) family. CS1 is a member of the CD2 subset of the immunoglobulin superfamily. Molecules of the CD2 family are involved in a broad range of immunomodulatory functions, such as co-activation, proliferation differentiation, and adhesion of lymphocytes, as well as immunoglobulin secretion, cytokine production, and NK cell cytotoxicity. Several members of the CD2 family, such as CD2, CD58, and CD150, play a role or have been proposed to play a role in a number of autoimmune and inflammatory diseases, such as psoriasis, rheumatoid arthritis, and multiple sclerosis. It has been reported that CS1 plays a role in NK cell-mediated cytotoxicity and lymphocyte adhesion (Bouchon, A. et al., J. Immunol., 5517-5521 (2001); Murphy, J. et al., Biochem. J., 361:431-436 (2002)).

Elotuzumab (trade name Empliciti, Bristol-Myers Squibb) is a humanized monoclonal IgG1 antibody directed against CS-1, a cell surface glycoprotein, which is highly and uniformly expressed in multiple myeloma. Elotuzumab induces significant antibody-dependent cellular cytotoxicity (ADCC) against primary multiple myeloma cells in the presence of peripheral lymphocytes (Tai et al., Blood, 112:1329-1337 (2008)). Results of three studies that evaluated the safety and efficacy of this drug administered alone (Zonder et al., Blood, 120(3):552-559 (2012)), in combination with bortezomib (Jakubowiak et al., J. Clin. Oncol., 30(16):1960-1965 (Jun. 1, 2012)), or lenalidomide and low-dose dexamethasone (Lonial et al., J. Clin. Oncol., 30:1953-1959 (2012); and Richardson et al., Blood (ASH Annual Meeting Abstracts), 116:986 (2010) for the treatment of patients with relapsed or refractory multiple myeloma, have been reported. All three combinations showed a manageable safety profile and encouraging activity (H. Magen and E. Muchtar. Ther Adv Hematol. 2016 August; 7 (4): 187-195).

The methods of the invention may include administration of other chemotherapeutic agents or treatment with a second therapy (e.g., a therapeutic agent or therapy that is standard in the art). A “chemotherapeutic agent” is a chemical compound useful in the treatment of cancer. Other examples of chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™, Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin y1 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubcrcidin, ubenimcx, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamide; thiotcpa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™, Genentech); and (xi) pharmaceutically acceptable salts, acids or derivatives of any of the above.

### Immune Modulating Molecules

In certain embodiments, one or more immune modulating compounds can be administered as part of the treatment plan. The immune-modulating molecules comprise, but are not limited to cytokines, chemokines, lymphokines, NK cell stimulating factors, T cell co-stimulatory ligands, etc. An immune-modulating molecule positively and/or negatively influences the humoral and/or cellular immune system, particularly its cellular and/or non-cellular components, its functions, and/or its interactions with other physiological systems. The immune-modulating molecule may be selected from the group comprising cytokines, chemokines, macrophage migration inhibitory factor (MIF; as described, inter alia, in Bernhagen (1998), Mol Med 76(3-4); 151-61 or Metz (1997), Adv Immunol 66, 197-223), T-cell receptors or soluble MHC molecules. Such immune-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3 (1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Cytokines are defined by any factor produced by cells that affect other cells and are responsible for any of a number of multiple effects of cellular immunity. Examples of cytokines include but are not limited to the IL-2 family, interferon (IFN), IL-7, IL-10, IL-12, IL-15, IL-18, IL-1, IL-17, TGF and TNF cytokine families, and to IL-1 through IL-35, IFN-α, IFN-β, IFNγ, TGF-β, TNF-α, and TNFβ.

Chemokines, similar to cytokines, are defined as any chemical factor or molecule which when exposed to other cells are responsible for any of a number of multiple effects of cellular immunity. Suitable chemokines may include but are not limited to the CXC, CC, C, and CX₃C chemokine families and to CCL-1 through CCL-28, CXC-1 through CXC-17, XCL-1, XCL-2, CX3CL1, MIP-1b, IL-8, MCP-1, and Rantes.

Growth factors include any molecules which when exposed to a particular cell induce proliferation and/or differentiation of the affected cell. Growth factors include proteins and chemical molecules, some of which include: stem cell factors, GM-CSF, G-CSF, human growth factor and stem cell growth factor. Additional growth factors may also be suitable for uses described herein.

Also of interest are enzymes present in the lytic package that NK cells, cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin O and pneumolysin facilitate granzyme B-dependent apoptosis (Browne et al., Mol. Cell Biol. 19:8604, 1999).

### Pharmaceutical Therapeutics

The invention provides pharmaceutical compositions comprising adoptive cell therapeutics and/or the IL-15 superagonist and/or second or third therapeutic agents such as for example, cytokines, chemotherapeutics, and the like, for use as a therapeutic. In one aspect, the pharmaceutical compositions are administered systemically, for example, formulated in a pharmaceutically-acceptable buffer such as physiological saline. Preferable routes of administration include, for example, instillation into the bladder, subcutaneous, intravenous, intraperitoneal, intramuscular, intratumoral or intradermal injections that provide continuous, sustained or effective levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia or infectious diseases, although in certain instances lower amounts will be needed because of the increased specificity of the compound. A compound is administered at a dosage that enhances an immune response of a subject, or that reduces the proliferation, survival, or invasiveness of a neoplastic or, infected cell as determined by a method known to one skilled in the art.

### Formulation of Pharmaceutical Compositions

The administration of compositions embodied herein, is by any suitable means that results in a concentration of the therapeutic that, combined with other components, is effective in ameliorating, reducing, or stabilizing the cancer, e.g. myeloma. The composition may be provided in a dosage form that is suitable for parenteral (e.g., subcutaneous, intravenous, intramuscular, intravesicular, intratumoral or intraperitoneal) administration route. For example, the pharmaceutical compositions are formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts are initially determined by extrapolating from the amount of compound used in mice or non-human primates, as a skilled artisan recognizes it is routine in the art to modify the dosage for humans compared to animal models. For example, the dosage may vary from between about 1 μg compound/kg body weight to about 5000 mg compound/kg body weight; or from about 5 mg/kg body weight to about 4,000 mg/kg body weight or from about 10 mg/kg body weight to about 3,000 mg/kg body weight; or from about 50 mg/kg body weight to about 2000 mg/kg body weight; or from about 100 mg/kg body weight to about 1000 mg/kg body weight; or from about 150 mg/kg body weight to about 500 mg/kg body weight. For example, the dose is about 1, 5, 10, 25, 50, 75, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,600, 1,700, 1,800, 1,900, 2,000, 2,500, 3,000, 3,500, 4,000, 4,500, or 5,000 mg/kg body weight. Alternatively, doses are in the range of about 5 mg compound/Kg body weight to about 20 mg compound/kg body weight. In another example, the doses are about 8, 10, 12, 14, 16 or 18 mg/kg body weight. In embodiments whereby the N-803 is administered to a patient as part of the therapy, the fusion protein complex is administered at 0.5 mg/kg-about 10 mg/kg (e.g., 0.5, 1, 3, 5, 10 mg/kg). Of course, this dosage amount may be adjusted upward or downward, as is routinely done in such treatment protocols, depending on the results of the initial clinical trials and the needs of a particular patient.

Pharmaceutical compositions are formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical compositions embodied herein are administered parenterally by injection, infusion or implantation (subcutaneous, intravenous, intramuscular, intratumoral, intravesicular, intraperitoneal) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions comprising the fusion protein complex or the chemotherapeutic agent, for parenteral use are provided in unit dosage forms (e.g., in single-dose ampoules). Alternatively, the composition is provided in vials containing several doses and in which a suitable preservative may be added (see below). The composition is in the form of a solution, a suspension, an emulsion, an infusion device, or a delivery device for implantation, or is presented as a dry powder to be reconstituted with water or another suitable vehicle before use. Apart from the active agent that reduces or ameliorates a neoplasia or infectious disease, the composition includes suitable parenterally acceptable carriers and/or excipients. The active therapeutic agent(s) may be incorporated into microspheres, microcapsules, nanoparticles, liposomes for controlled release. Furthermore, the composition may include suspending, solubilizing, stabilizing, pH-adjusting agents, tonicity adjusting agents, and/or dispersing, agents.

As indicated above, the pharmaceutical compositions may be in a form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

The present invention provides methods of treating neoplasia or infectious diseases or symptoms thereof which comprise administering a therapeutically effective amount of a pharmaceutical composition. Thus, one embodiment is a method of treating a subject suffering from or susceptible to a neoplasia or infectious disease or symptom thereof. The method includes the step of administering to the mammal a therapeutic amount of the compositions embodied herein and the adoptive cell therapy sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a neoplasia, infectious disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The fusion protein complexes of the invention may be used in the treatment of any other disorders in which an increase in an immune response is desired.

### Kits or Pharmaceutical Systems

Pharmaceutical compositions comprising the therapeutic components embodied herein, such as adoptive cell therapy, IL-15 superagonist, chemotherapeutic agents, cytokines, etc., may be assembled into kits or pharmaceutical systems for use in treatment of myelomas or cancers in general. Kits or pharmaceutical systems according to this aspect of the invention comprise a carrier means, such as a box, carton, tube, having in close confinement therein one or more container means, such as vials, tubes, ampoules, bottles and the like. The kits or pharmaceutical systems of the invention may also comprise associated instructions for using the fusion protein complex of the invention. In one embodiment, the kit includes appropriate containers such as bags, bottles, tubes, to allow ex vivo treatment of immune cells using the fusion protein complex of the invention and/or administration of such cells to a patient. Kits may also include medical devices comprising the fusion protein complex of the invention.

### EXAMPLES

### Example 1: Combination Therapies

To produce cells for adoptive therapy treatments or bone marrow transplantion (BMT), experiments are conducted using mice. Female mice, such as C57BL/6 (B6, H-2K^{b}), Balb/c (H-2K^{d}), B6CBAF1 (H-2K^{b/k}), CB6F1 (H-2K^{b/d}) and B6D2F1 (H2K^{b/d}) are obtained from the Jackson Laboratory (Bar Harbor, ME). Mice for use in BMT experiments are between 10-12 weeks of age.

Bone marrow (BM) cells are removed aseptically from femurs and tibias and T cells are depleted (TCD) by incubation with anti-Thy 1.2 antibody for 30 min at 4° C., followed by incubation with Low-TOX-M rabbit complement (Cedarlane Laboratories, Hornby, Ontario, Canada) for 40 minutes at 37° C., or alternatively via anti-CD5 magnetic bead depletion (Miltenyi, Auburn, CA). Typical levels of contaminating T cells after complement depletion range from 0.2 to 0.5 percent of all bone marrow leukocytes.

Splenic T cells are obtained by positive selection with anti-CD5 antibodies conjugated to magnetic beads (Miltenyi, Auburn, CA). In some cases, CD4+and CD8+T cell populations are separated out individually (Miltenyi, Auburn, CA). Cells (5×10⁶ BM cells with or without splenic T cells) are resuspended in Dulbecco Modified Eagle's Medium (DMEM) and are transplanted by tail vein infusion (0.25 ml total volume) into lethally irradiated recipients on day 0. On day 0 prior to transplantation, recipients receive 11 to 13 Gy total body irradiation (strain dependent) from a ¹³⁷Cs source as a split dose with a 3 hour interval between doses to reduce gastrointestinal toxicity. Mice will be housed in sterilized micro-isolator cages and will receive normal chow and autoclaved hyper-chlorinated drinking water (pH 3.0).

Cell lines, Antibodies, and Cytokines: The P-815 (H-2d) cell line will be obtained from ATCC (Manassas, VA). Cells are cultured in RPMI with 10% FBS in atmosphere containing 5% CO_{2.}

Anti-murine CD16/CD32 FcR block (2.4G2) and all of the following fluorochrome-labeled antibodies against murine antigens will be obtained from BD Pharmingen (San Diego, CA): H2Kd (SF1-1.1), CD3 (500A2), CD4 (RM4-5), CD8 (53-6.7), CD25 (PC61), CD44 (IM7), CD45R/B220 (RA3-6B2), CD62L (MEL-14), NK1.1 (PK136), TNF-α (MP6-XT22), IFN-γ (XMG1.2), NK2GD, isotype controls; rat IgG2a-, rat IgG1-K hamster, and IgG1-κ.

The IL-15 superagonist, N-803 is generated by Altor BioScience Corporation, Miramar, Florida. N-803 will be administered intraperitoneally, weekly at 1-5 μg/day.

Elotuzumab (EMPLICITI™) can be obtained from Bristol Myers Squibb and will follow the recommended dosage initially.

Flow Cytometry: Single cell suspensions of 10⁶ cells/25 μL are incubated at 4° C. with CD16/CD32 FcR block. Subsequently, cells are incubated at 4° C. with antibodies in a total volume of 50μl. The stained cells are analyzed on a FACS Calibur flow cytometer (Becton Dickinson, San Jose, CA) with CellQuest software or LSRII cytometer (Becton Dickinson, San Jose, CA) with FlowJo software (Treestar, San Carlos, CA).

Assessment of Graft-Versus-Host-Disease: The severity of GVHD will be assessed with a clinical GVHD scoring system as previously described (Cooke, K. R., et al., Blood, 1996. 88(8): p. 3230-9). Briefly, ear-punched animals in coded cages are individually scored every week using 5 clinical parameters based on a scale from 0 to 2: weight loss, posture, mobility, fur, and skin. A clinical GVHD index is generated by summation of the 5 criteria scores (0-10). Survival is monitored daily. Animals with scores of at least 5 are considered moribund and will be sacrificed.

PMA-Ionomycin Stimulation and Intracellular Staining: Splenocytes will be incubated with PMA (20 ng/mL) and ionomycin (1 μM) for 5 hours. Brefeldin A is added at a concentration of 10 μg/mL two hours following the addition of PMA and ionomycin. Cells are first stained with surface antibodies and then fixed and permeabilized with the BD Cytofix/Cytoperm Kit (BD Biosciences, San Diego, CA) and subsequently stained with intracellular antibodies.

CFSE Labeling: Cells are labeled with carboxyfluorescein succinimidyl ester (CFSE) as previously described (Lyons, A. B. and C. R. Parish, Determination of lymphocyte division by flow cytometry. J Immunol Methods, 1994. 171(1): p. 131-7). Briefly, splenocytes are incubated with CFSE at a final concentration of 2.5 μM in PBS at 37° C. for 20 minutes. Cells are then washed three times with PBS before intravenous injection.

Combination Therapies: The effects of component of the therapies individually and in combination will be conducted on cells in vitro followed by in vivo experiments. The immune function of the various immune effector cells will be assessed prior to and at intervals after administration of the adoptive cell therapy, IL-15 superagonist and Elotuzumab.

Statistics: All values will represent the mean±SEM. Survival data will be analyzed using the Mantel-Cox log-rank test. For all other analysis, nonparametric unpaired Mann-Whitney-U test will be used.

### Other Embodiments

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed:

1. A method of treating cancer, comprising:
administering to a subject suffering from a cancer an effective amount of: i) an adoptive cell therapy, ii) an IL-15:IL-15Rα complex, and iii) at least one chemotherapeutic agent,
thereby treating the cancer.

2. The method of claim 1, wherein the IL-15/IL15Rα complex is an IL-15N72D:IL-15RαSu/Fc complex comprising a dimeric IL-15RαSu/Fc and two IL-15N72D molecules.

3. The method of claim 1 or 2, wherein the adoptive cell therapy comprises hematopoietic stem cell transplantation, donor leukocyte infusion, adoptive transfer of natural killer cells (NK), T cells, B cells, chimeric antigen receptor-T cells (CAR-T), chimeric antigen receptor natural killer cells (CAR-NK) or combinations thereof.

4. The method of any one of claims 1 through 3, wherein the adoptive cell therapy comprises NK cells.

5. The method of any one of claims 1 through 4, wherein the adoptive cell therapy comprises transfer of allogeneic, autologous, syngeneic, related, unrelated, HLA-matched, HLA-mismatched or haploidentical cells.

6. The method of any one of claims 1 through 5, wherein the cancer comprises:
myeloma, multiple myeloma, smoldering myeloma, relapsed or refractory multiple myeloma, hematological cancer, chronic myelogenous leukemia, acute myelogenous leukemia, acute lymphoblastic leukemia, myelodysplasia, mantle cell lymphoma, B cell non-Hodgkin lymphoma, Hodgkin's lymphoma, chronic lymphocytic leukemia, lymphoma, non-Hodgkin's lymphomas (NHL), chronic lymphocytic leukemia, follicular lymphoma, mantle cell lymphoma or diffuse large B-cell lymphoma.

7. The method of any one of claims 1 through 5, wherein the cancer comprises myeloma, multiple myeloma, smoldering myeloma, relapsed or refractory multiple myeloma.

8. The method of any one of claims 1 through 7, wherein the chemotherapeutic agent comprises: anti-CS1 antibody (Elotuzumab), bortezomib, lenalidomide (Revlimid), dexamethasone, melphalan, vincristine (Oncovin), cyclophosphamide (Cytoxan), etoposide (VP-16), doxorubicin (Adriamycin), liposomal doxorubicin (Doxil), bendamustine (Treanda), anti-PD1 antibody (nivolumab or pembrolizumab) or combinations thereof.

9. The method of any one of claims 1 through 8, wherein the chemotherapeutic agent comprises anti-CS1 antibody (Elotuzumab).

10. The method of any one of claims 1 through 9, wherein the at least one chemotherapeutic agent is administered prior to, simultaneously with, sequentially to the adoptive cell therapy, or any combination thereof.

11. The method of any one of claims 1 through 9, wherein the at least one chemotherapeutic agent is administered prior to the administration of the adoptive cell therapy.

12. The method of any one of claims 1 through 9, wherein the at least one chemotherapeutic agent is administered concomitantly with the administration of the adoptive cell therapy.

13. The method of any one of claims 1 through 9, wherein the at least one chemotherapeutic agent is administered after the administration of the adoptive cell therapy.

14. The method of any one of claims 1 through 13, further comprising administering an immunomodulatory agent, anti-anemia agents, radiation therapy, corticosteroids, cytokines, chemokines or combinations thereof.

15. The method of any one of claims 1 through 14, wherein a therapeutically effective amount of the IL-15N72D:IL-15RαSu/Fc complex is administered once or twice per week.

16. The method of any one of claims 1 through 14, wherein a therapeutically effective amount of the IL-15N72D:IL-15RαSu/Fc complex is administered daily.

17. The method of any one of claims 1 through 16, wherein a therapeutically effective amount of the IL-15N72D:IL-15RαSu/Fc complex is between 0.1 μg/Kg and 100 mg/Kg.

18. The method of any one of claims 1 through 17 wherein the pharmaceutical composition is administered systemically, intravenously, subcutaneous, intramuscularly, intravesically, or by instillation.

19. The method of claim any one of claims 1 through 18 wherein the IL-15:IL-15Rα complex stimulates proliferation or activation of adoptively transferred cells.

20. A pharmaceutical composition comprising an effective mount of adoptive cell therapy, an IL-15/IL-15Rα fusion complex, a chemotherapeutic agent or combinations thereof.

21. The pharmaceutical composition of claim 20, wherein the IL-15/IL-15Rα fusion complex is IL-15N72D:IL-15RαSu/Fc.

22. The pharmaceutical composition of claim 20 or 21, wherein the chemotherapeutic agent comprises: anti-CS1 antibody (Elotuzumab), bortezomib, lenalidomide (Revlimid), dexamethasone, melphalan, vincristine (Oncovin), cyclophosphamide (Cytoxan), etoposide (VP-16), doxorubicin (Adriamycin), liposomal doxorubicin (Doxil), bendamustine (Treanda), anti-PD1 antibody (nivolumab or pembrolizumab) or combinations thereof.

23. A pharmaceutical composition comprising an effective amount of IL-15/IL-15Rα and a chemotherapeutic agent comprising: anti-CS1 antibody (Elotuzumab), bortezomib, lenalidomide (Revlimid), dexamethasone, melphalan, vincristine (Oncovin), cyclophosphamide (Cytoxan), etoposide (VP-16), doxorubicin (Adriamycin), liposomal doxorubicin (Doxil), bendamustine (Treanda), anti-PD1 antibody (nivolumab or pembrolizumab) or combinations thereof.

24. The pharmaceutical composition of claim 23, wherein the fusion complex is IL-15N72D:IL-15RαSu/Fc.

25. The pharmaceutical composition of claim 23 or 24, wherein the chemotherapeutic agent is an anti-CS1 antibody (Elotuzumab).

26. A method of treating a myeloma comprising: administering to a subject an effective amount of: i) an adoptive cell therapy, ii) an IL-15:IL-15Rα complex, and iii) at least one chemotherapeutic agent,
thereby treating the myeloma.

27. The method of claim 26, wherein the IL-15/IL15Rα complex is an IL-15N72D:IL-15RαSu/Fc complex comprising a dimeric IL-15RαSu/Fc and two IL-15N72D molecules.

28. The method of claim 26 or 27, wherein the adoptive cell therapy comprises hematopoietic stem cell transplantation, donor leukocyte infusion, adoptive transfer of natural killer cells (NK), T cells, B cells, chimeric antigen receptor-T cells (CAR-T), chimeric antigen receptor natural killer cells (CAR-NK) or combinations thereof.

29. The method of any one of claims 26 through 28, wherein the chemotherapeutic agent comprises: anti-CS1 antibody (Elotuzumab), bortezomib, lenalidomide (Revlimid), dexamethasone, melphalan, vincristine (Oncovin), cyclophosphamide (Cytoxan), etoposide (VP-16), doxorubicin (Adriamycin), liposomal doxorubicin (Doxil), bendamustine (Treanda), anti-PD1 antibody (nivolumab or pembrolizumab) or combinations thereof.

30. The method of any one of claims 26 through 28, wherein the chemotherapeutic agent is elotuzumab.

31. A kit for treating cancer, the kit comprising an adoptive cell therapy, an IL-15/IL15Rα complex, and at least one chemotherapeutic agent and directions for the use of the kit for the treatment of cancer.

32. A kit for treating myeloma, the kit comprising an adoptive cell therapy, an IL-15/IL15Rα complex, and at least one chemotherapeutic agent and directions for the use of the kit for the treatment of myeloma.

33. The kit of claim 31 or 32, wherein said adoptive cell therapy comprises hematopoietic stem cells, donor leukocytes, T cells, or natural killer (NK) cells.

34. The kit of any one of claims 31 through 33, wherein the IL-15/IL15Rα complex is an IL-15N72D:IL-15RαSu/Fc complex comprising a dimeric IL-15RαSu/Fc and two IL-15N72D molecules.

35. The kit of any one of claims 31 through 34, wherein the chemotherapeutic agent is an anti-CS1 antibody (Elotuzumab).
